(19) United States
(12) Patent Application Publication
(10) Pub. No.: US 2018/0308576 A1
(43) Pub. Date: Oct. 25, 2018

(54) SYSTEM AND METHOD FOR PATIENT TRACKING DURING MASS CASUALTY EVENTS

(71) Applicant: Dallas/Fort Worth International Airport Board, DFW Airport, TX (US)

(72) Inventor: David Young, Grand Prairie, TX (US)

(21) Appl. No.: 15/955,456

(22) Filed: Apr. 17, 2018

Related U.S. Application Data
(60) Provisional application No. 62/488,507, filed on Apr. 21, 2017.

(51) Int. Cl. G16H 40/20; G16H 10/60; H04W 4/90

(52) U.S. Cl. CPC G16H 40/20; H04W 4/90; G16H 10/60

(57) ABSTRACT

A method includes sending a broadcast notification message concurrently to multiple local medical facilities about a mass casualty event at an event site. The method also includes obtaining, from each of the multiple medical facilities in response to the broadcast notification message, available bed count information of the medical facility and storing the available bed count information of each medical facility in a database. The method further includes receiving patient data for each of a plurality of patients involved in the mass casualty event and storing the patient data in the database. In addition, the method includes displaying at least some of the patient data and at least some of the available bed count information on a patient tracking board and a hospital tracking display at a user interface.

### CROSS-REFERENCE TO RELATED APPLICATION AND PRIORITY CLAIM

This application claims priority under 35 U.S.C. § 119(e) to U.S. Provisional Patent Application No. 62/488,507 filed on Apr. 21, 2017 and entitled “SYSTEM AND METHOD FOR PATIENT TRACKING DURING MASS CASUALTY EVENTS,” the contents of which are hereby incorporated by reference in their entirety.

### TECHNICAL FIELD

This disclosure is generally directed to a system and method for patient tracking during mass casualty events at large venues, such as at airports, other transportation terminals, government buildings, sports venues, concerts, and the like.

### BACKGROUND

Patient tracking during mass casualty events has been identified as one of the most challenging aspects of accident and emergency response. A large number of “solutions” are readily available in the market, but field testing of these solutions under real-world conditions has identified a large number of deficiencies and failings.

### SUMMARY

This disclosure provides a system and method for patient tracking during mass casualty events at large venues. While the disclosed embodiments are described in conjunction with an emergency that occurs at an airport, this is merely one example. The disclosed embodiments are also applicable for the site of a crash or accident involving a large number of people or vehicles, such as an airplane crash, a bus or train accident, a building fire or explosion, and the like. The disclosed embodiments are also applicable for other events in other venues with large numbers of people, such as other transportation terminals (e.g., train terminals), government buildings, shopping centers, sports venues, concerts, and the like.

In a first embodiment, a method includes sending a broadcast notification message concurrently to multiple local medical facilities about a mass casualty event at an event site. The method also includes obtaining, from each of the multiple medical facilities in response to the broadcast notification message, available bed count information of the medical facility and storing the available bed count information of each medical facility in a database. The method further includes receiving patient data for each of a plurality of patients involved in the mass casualty event and storing the patient data in the database. In addition, the method includes displaying at least some of the patient data and at least some of the available bed count information on a patient tracking board and a hospital tracking display at a user interface.

In a second embodiment, an apparatus includes at least one processing device and a display configured to show a user interface. The at least one processing device is configured to send a broadcast notification message concurrently to multiple local medical facilities about a mass casualty event at an event site. The at least one processing device is also configured to obtain, from each of the multiple medical facilities in response to the broadcast notification message, available bed count information of the medical facility and store the available bed count information of each medical facility in a database. The at least one processing device is further configured to receive patient data for each of a plurality of patients involved in the mass casualty event and store the patient data in the database. In addition, the at least one processing device is configured to control the display to display at least some of the patient data and at least some of the available bed count information on a patient tracking board and a hospital tracking display at the user interface.

In a third embodiment, a non-transitory computer readable medium contains instructions that when executed cause at least one processing device to send a broadcast notification message concurrently to multiple local medical facilities about a mass casualty event at an event site. The medium also contains instructions that when executed cause the at least one processing device to obtain, from each of the multiple medical facilities in response to the broadcast notification message, available bed count information of the medical facility and store the available bed count information of each medical facility in a database. The medium further contains instructions that when executed cause the at least one processing device to receive patient data for each of a plurality of patients involved in the mass casualty event and store the patient data in the database. In addition, the medium contains instructions that when executed cause the at least one processing device to control a display to display at least some of the patient data and at least some of the available bed count information on a patient tracking board and a hospital tracking display at a user interface.

Other technical features may be readily apparent to one skilled in the art from the following figures, descriptions, and claims.

### BRIEF DESCRIPTION OF THE DRAWINGS

For a more complete understanding of this disclosure and its features, reference is now made to the following description, taken in conjunction with the accompanying drawings, in which:

FIG. 1 illustrates an example system supporting patient tracking during a mass casualty event according to this disclosure;

FIG. 2 illustrates an example user interface of an automated notification system according to this disclosure;

FIG. 3 shows an example user interface in which an officer may enter bed counts at a medical facility, according to this disclosure;

FIGS. 4A through 4E illustrate portions of a patient tracking form in which patient data can be entered according to this disclosure;

FIG. 5 illustrates an example patient tracking board according to this disclosure;

FIG. 6 illustrates an example “green” tracking patient board according to this disclosure;

FIG. 7 illustrates an example patient tracking display according to this disclosure;

FIG. 8 illustrates an example device for patient tracking during a mass casualty event according to this disclosure; and

FIG. 9 illustrates an example method for patient tracking during a mass casualty event according to this disclosure.

### DETAILED DESCRIPTION

The figures discussed below and the various embodiments used to describe the principles of the present invention in this patent document are by way of illustration only and should not be construed in any way to limit the scope of this disclosure. Those skilled in the art will understand that the principles of the present invention may be implemented in any type of suitably arranged device or system.

The disclosed embodiments provide an emergency management solution for patient tracking during mass casualty events. The disclosed embodiments were created to solve key problems identified by the National Transportation Safety Board (NTSB) in mass casualty events. The disclosed embodiments advantageously speed up the tracking of patients, provide a superior information display from real-time data to enhance decision making, and minimize the loss of tracking on patients admitted outside a Main Triage Control Point. At least some components of the disclosed embodiments can be developed on a simple, collaborative, and open platform (such as G-Suites by GOOGLE or any other suitable platform), which enhances the ability to customize the solution and implement the solution in many environments.

It will be understood that embodiments of this disclosure may include any one, more than one, or all of the features described here. In addition, embodiments of this disclosure may additionally or alternatively include other features not listed here. Some of the following embodiments are described with respect to an emergency that occurs at an airport. However, such description is not limiting; it will be clear to those of skill in the art that the disclosed embodiments are also applicable in other venues with large numbers of people, such as other transportation terminals (e.g., train terminals), government buildings, sports venues, concerts, and the like.

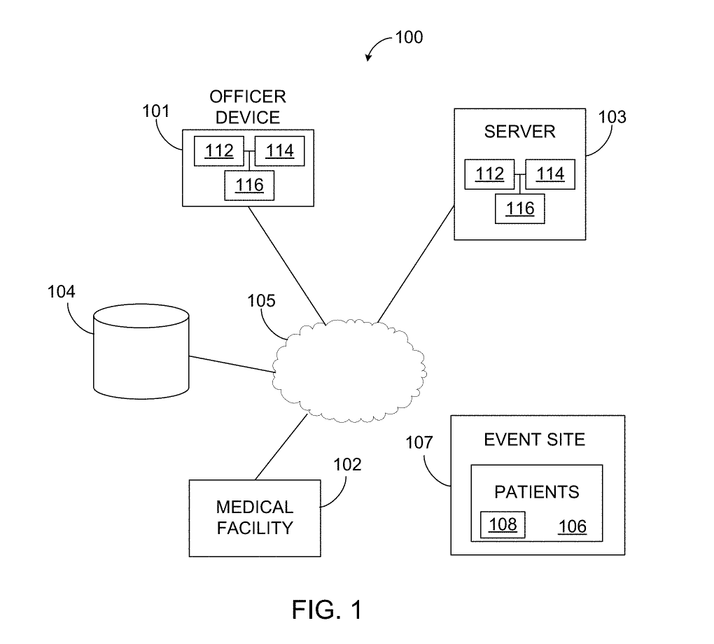

FIG. 1 illustrates an example system 100 supporting patient tracking during a mass casualty event according to this disclosure. As shown in FIG. 1, the system 100 includes an officer device 101, a medical facility 102, a server 103, and a database 104. The officer device 101 can be used by an emergency medical services officer or other first responder at the site 107 of the mass casual event to collect and enter data associated with one or more patients 106 that have been involved in the mass casualty event, such as an emergency at an airport or on an airplane. Such patient data can include name and demographic information, triage category, types of injury, and the like. The patient data can also include identifying or descriptive information about the patient, such as hair color, clothing worn, or distinctive features (e.g., the presence of a cross tattoo on the left forearm). The officer device 101 includes any suitable device for collecting and entering data associated with one or more patients. In some embodiments, the officer device 101 can include a wireless communication device or wireless computing device, such as a smart phone, tablet, or laptop computer. In some embodiments, the officer device 101 can include a scanner, RFID reader, or other data reader to scan and read information from a barcode, RFID tag, or other device 108 worn by each patient 106. While FIG. 1 shows one officer device 101, it will be understood that the system 100 can include multiple officer devices 101.

The medical facility 102 represents a hospital, emergency center, urgent care center, doctor's office, or other facility that provides medical care (especially urgent care or emergency care) to patients. The medical facility 102 can include hospital beds, a trauma center, medical personnel, and any other elements required to treat patients in the event of an emergency. The medical facility 102 can also include computing devices, network devices, servers, or other equipment that can transmit and receive information to/from the officer device 101, the server 103, and the database 104. While FIG. 1 shows one medical facility 102, it will be understood that the system 100 can include multiple medical facilities 102.

The server 103 and the database 104 are used in the system 100 to support the patient tracking. For example, the database 104 can be used to store information provided by the officer device 101 and/or medical facility 102, and the server 103 can retrieve and present the information on the officer device 101 and/or at the medical facility 102. The server 103 and the database 104 could support any other or additional activities for patient tracking. The server 103 includes any suitable computing device(s) supporting patient tracking. The database 104 includes any suitable device(s) for storing and facilitating retrieval of information. While FIG. 1 shows one server 103 and one database 104, it will be understood that the system 100 can include multiple servers 103 and multiple databases 104. In some embodiments, each medical facility 102 could have at least one server 103 or at least one database 104. In other embodiments, one server 103 or group of servers 103 and one database 104 or group of databases 104 could support data and applications for the entire system 100.

The officer device 101, medical facility 102, server 103, and database 104 are coupled to at least one network 105. Each network 105 facilitates communication between various components coupled to the network. For example, a network 105 can communicate Internet Protocol (IP) packets, frame relay frames, Asynchronous Transfer Mode (ATM) cells, or other suitable information between network addresses. The network(s) 105 can include one or more local area networks, metropolitan area networks, wide area networks, all or a portion of a global network, or any other communication system(s) at one or more locations.

In this example, each officer device 101 and server 103 could include at least one processing device 112, such as at least one microprocessor, microcontroller, digital signal processor, or other processing or control device(s). Each officer device 101 and server 103 could also include at least one memory 114 for storing and facilitating retrieval of information used, generated, or collected by the processing device(s) 112. Each officer device 101 and server 103 could further include at least one network interface 116 configured to support communications over at least one network, such as a wired network interface (like an Ethernet interface) or a wireless network interface (like a radio frequency transceiver).

Communications between and amongst the various components shown in FIG. 1 could occur using any suitable physical or wireless communication media. For example, each device shown in FIG. 1 could include at least one interface for communicating over physical or wireless communication links. Each device shown in FIG. 1 could include any suitable interface or combination of interfaces.

Although FIG. 1 illustrates one example of a system 100 supporting patient tracking during a mass casualty event, various changes can be made to FIG. 1. For example, the system 100 could include any number of officer devices, medical facilities, networks, servers, and databases in any suitable configuration(s). Also, the functional division shown in FIG. 1 is for illustration only. Various components in FIG. 1 could be combined, further subdivided, or omitted and additional components could be added according to particular needs. For instance, the functionality of the server 103 and/or database 104 could be incorporated into each officer device 101 and/or each medical facility 102.

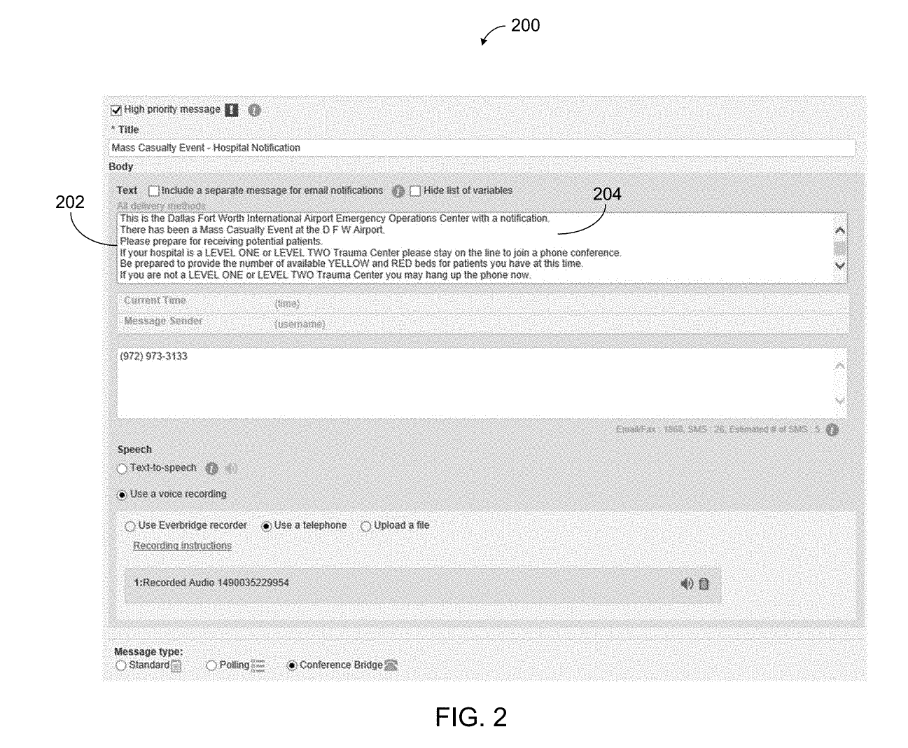

FIGS. 2 through 7 illustrate aspects of a process for patient tracking during a mass casualty event according to this disclosure. An early step of the process (which will now be referred to as “step one”) is activation of an automated notification system that alerts all medical facilities in the local area of the mass casualty event. The automated notification system is part of the system 100 and can be activated on a computing device or communication device, such as the officer device 101 of FIG. 1. Typically, the device is operated by a worker (e.g., a medical ops officer) on the scene of the mass casualty event.

FIG. 2 illustrates an example user interface 200 of the automated notification system according to this disclosure. The user interface 200 displays an editable box 202 that contains a notification message 204. The automated notification system is preconfigured with a list of medical facilities 102 (especially critical Level 1 and Level 2 trauma centers) in the area and their contact information. Using the preconfigured list, the notification message 204 is automatically and concurrently broadcast to the medical facilities 102 in the area. This allows multiple medical facilities 102 to prepare for, and also be looking for, mass casualty patients. In some embodiments, the broadcast can be in the form of a conference call that includes the broadcasting device (e.g., the officer device 101) and the medical facilities 102. In other embodiments, the broadcast can be in the form of a textual network message (e.g., text message, email, etc.). It is from this broadcast that the medical ops officer can receive accurate bed counts from the medical facilities 102. That is, the broadcast gives each medical facility 102 an opportunity to reply with bed count information, either verbally or through a written communication (e.g., a return text message or email).

The automatic and concurrent notifications of the automated notification system can easily be performed with just a few button presses on a wireless communication device, such as a smartphone, since the entire message and distribution list are all predetermined when the automated notification system is activated on the device. This notification step of the process facilitates rapid communication and exchange of information between multiple stakeholders, which is critical in a mass casualty event. This represents a technical advantage over conventional systems that require a medical ops officer calling each medical facility individually from the field via a cell phone or other phone.

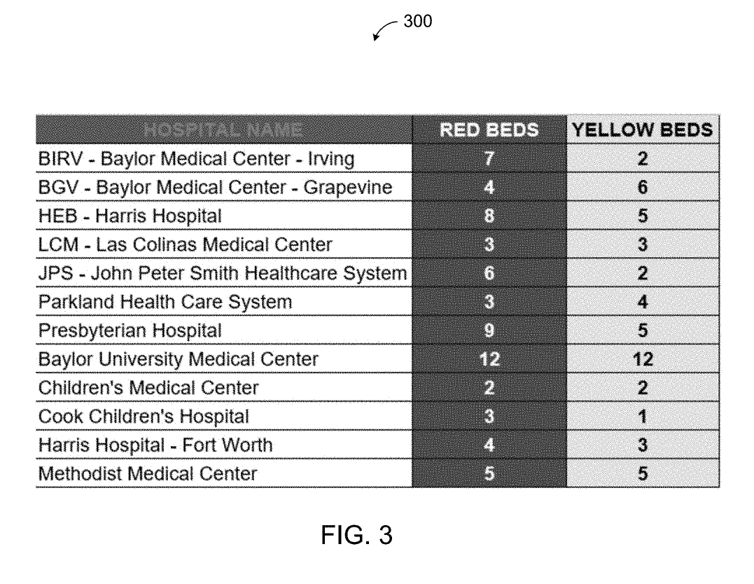

Step two of the process is tracking the available bed counts provided from each of the medical facilities 102 in the automated notification system of step one. For example, FIG. 3 shows an example user interface 300 in which a medical ops officer may enter, update, or review the bed counts, according to this disclosure. The user interface 300 can be displayed on the officer device 101 of FIG. 1 or another suitable computing device. The bed count for each medical facility 102 can include red beds and yellow beds (where red and yellow represent different levels of care). In some embodiments, this is the only data that the officer has to enter in the user interface 300. The bed count data can be entered by the officer based on information received during the conference call. Alternatively, in embodiments where a data connection is formed with the medical facilities 102, the bed count data can be automatically downloaded from each medical facility 102, and the medical ops officer does not need to enter any information. For example, in some embodiments, during step one, a hyperlink is automatically generated and displayed to each medical facility 102. By clicking on the hyperlink, personnel at the medical facility 102 can one-click into the system 100 and enter their current bed counts. Later, as patients are received at the medical facility 102, the medical personnel can again click on the hyperlink to update their current bed counts. Additionally or alternatively, some cities, municipalities, and hospitals have a corresponding system where this information is currently being tracked and updated. In such cases, the system 100 can automatically interface with the corresponding system to retrieve the information.

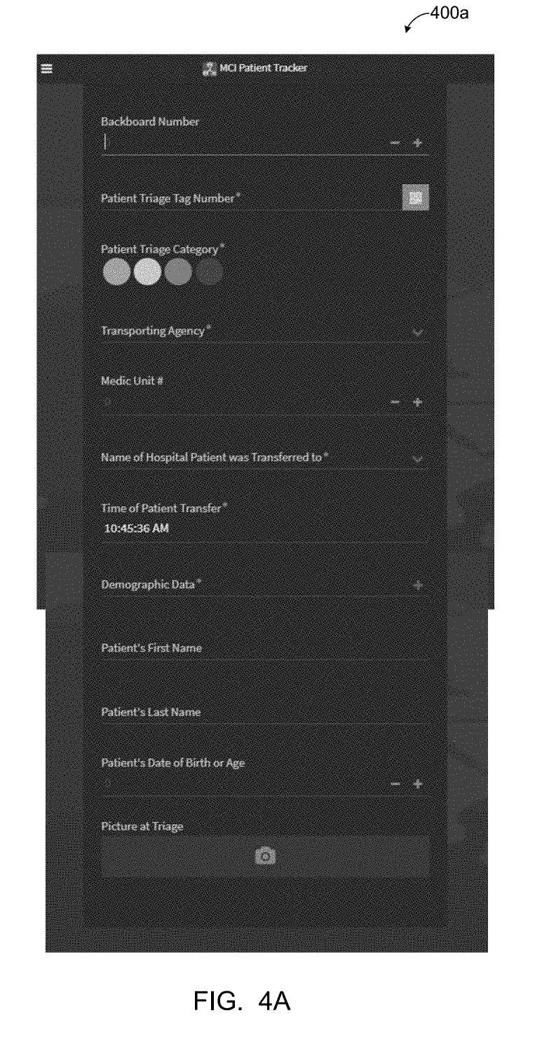

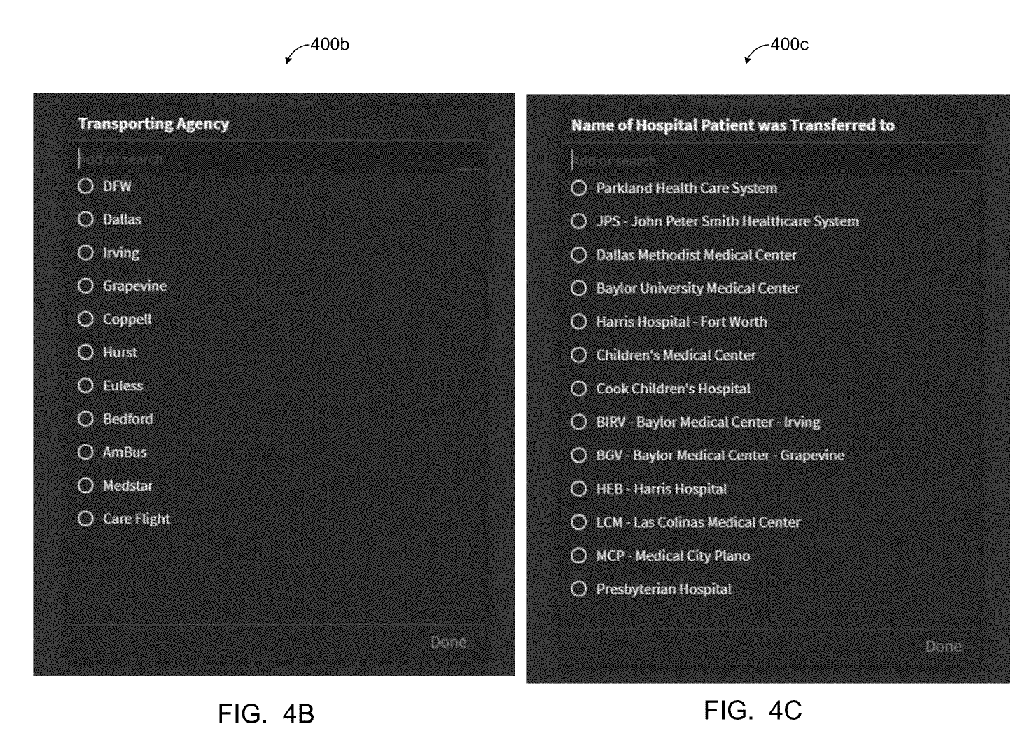

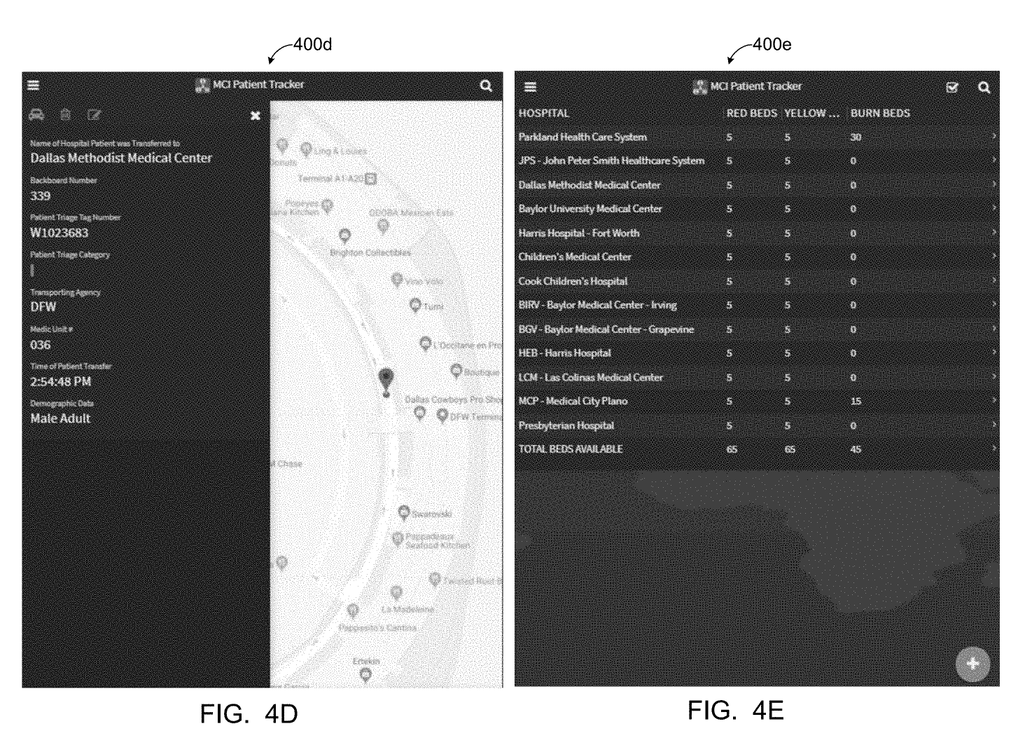

The third step of the process is to enter patient data in the system 100. FIGS. 4A through 4E illustrate portions 400a-400e of a patient tracking form 400 in which patient data can be entered according to this disclosure. The portions 400a-400e of the patient tracking form 400 can be displayed together, separately, or sequentially on the officer device 101 of FIG. 1 or another suitable computing device. As shown in FIGS. 4A through 4E, such patient data can include name and demographic information, triage category, types of injury, transporting agency, hospital, and the like. The patient data can also include identifying or descriptive information about the patient, such as hair color, clothing worn, or distinctive features (e.g., the presence of a cross tattoo on the left forearm). In some embodiments, the entry of the patient data can be facilitated by scanning a barcode, RFID tag, or other information source. For example, the medical ops officer can use the officer device 101 to scan a barcode 108 on a wristband or other tag provided to and worn by each patient 106, such as in a triage location. Data read from the barcode can be automatically loaded into the patient tracking form 400, and then sent to a server 103 or a database 104 for storage and processing.

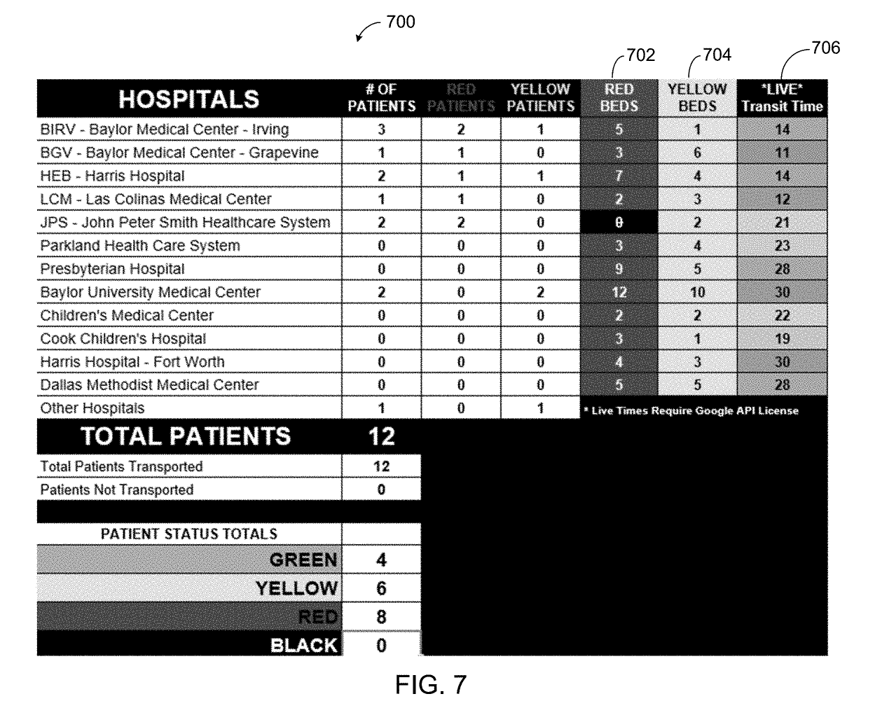

The patient tracking form 400 is compliant with HIPAA (Health Insurance Portability and Accountability Act) regulations (e.g., compliant with 42 C.F.R. § 403.812). The patient tracking form 400 is also streamlined so that only the most critical information must be entered to determine who went where, when did they go, and how did they get there. As shown in FIGS. 4A through 4E, much of the patient tracking form 400 can include touch-activated control buttons (e.g., check boxes or radio buttons) for easy entry of patient data. Information for the control buttons can be preconfigured in the patient tracking form 400 from data stored in, and retrieved from, the database 104. The presence of the control buttons in the patient tracking form 400 serve as reminders to the user to provide all necessary or important information. All of the required data in the patient tracking form 400 can be entered with only a few taps on a screen. Once the patient data is entered in the patient tracking form 400, the patient data can be used immediately to populate other forms or user interfaces, such as the patient tracking display 700 (described below with respect to FIG. 7). The information in the patient tracking form 400 provides both the line-by-line patient tracking desired by command staff and outside stakeholders, such as airlines, but also can be parsed out into visual data better utilized by on-scene staff to make life saving decisions.

Because the system 100 is network-enabled and collaborative, population of the patient tracking form 400 in the third step of the process can be performed simultaneously by multiple users on multiple officer devices 101, and collected and stored centrally in the database 104. In some embodiments, when connectivity is temporarily unavailable, patient information can be entered and stored locally in a memory of the officer device 101. Later, when the officer device 101 is connected to the network 105, it can be downloaded from the officer device 101 to the server 103, the database 104, or other information repository.

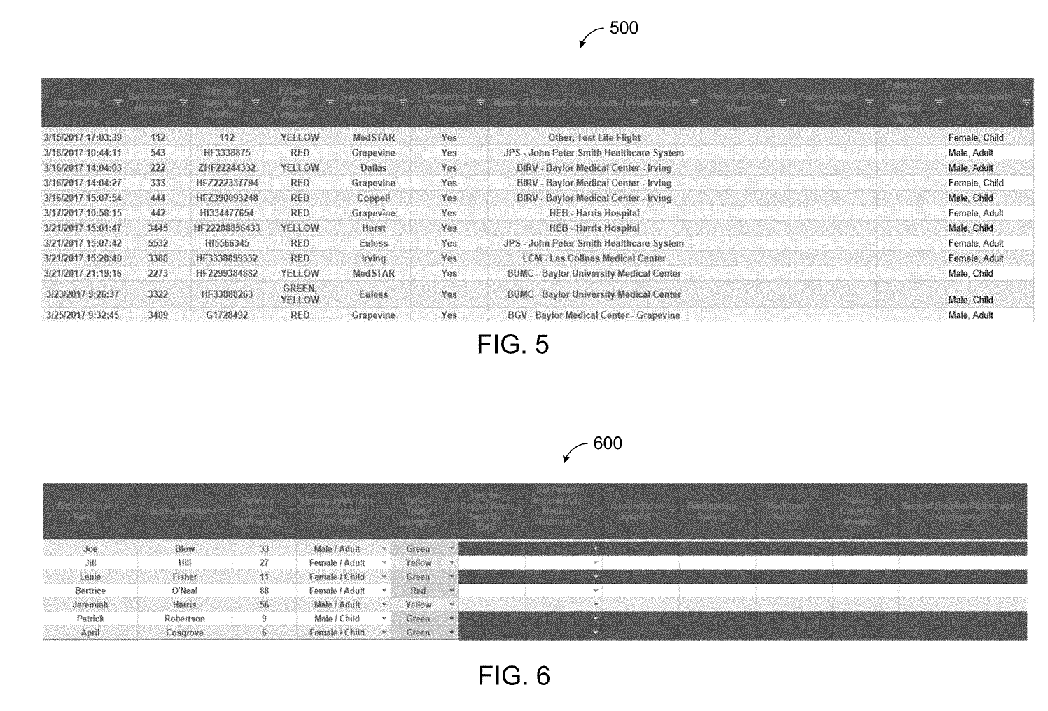

FIG. 5 illustrates an example patient tracking board 500 according to this disclosure. The patient tracking board 500 can be displayed on the officer device 101 of FIG. 1 or another suitable computing device. The patient tracking board 500 provides a dynamically generated summary view of real time patient data for patients involved in the mass casualty event. Information shown in the patient tracking board 500 can be retrieved from the database 104 and can include information from the patient tracking forms 400. For example, as patient tracking forms 400 are submitted for each patient in the field, the patient data from the patient tracking forms 400 can be stored in the database 104 and automatically propagated in real time into the patient tracking board 500. The patient tracking board 500 allows emergency operations center (EOC) representatives to see patient data in real time. This is valuable so that EOC representatives can contact the correct hospitals or other medical facilities to get patient data that can be matched against items like passenger manifest lists.

FIG. 6 illustrates an example “green” tracking patient board 600 according to this disclosure. The “green” patient tracking board 600 can be displayed on the officer device 101 of FIG. 1 or another suitable computing device. The “green” tracking patient board 600 tracks “green” (walking minor wounded or uninjured) patients as they are processed to a Passenger Gathering Center or other facility. According to the NTSB, many people who do not appear to be injured at first often discover injuries as adrenaline wears off. These patients are then transported via ambulance to a hospital or other medical facility, but because they did not go through the standard field triage, they are not tracked and are “lost” in the system, often times for days. The “green” tracking patient board 600 provides a method to gather basic data about “green” patients that are gathered at a Passenger Gathering Center or other facility.

If a patient is a “green” patient, the “green” tracking patient board 600 allows the collection of basic demographic data. Later, if the patient experiences a medical emergency like neck or back pain or cardiac arrest, the patient's status can be switched to yellow or red, which activates additional cells or fields in the “green” tracking patient board 600 to enter transportation and hospital data. Thus, the patient's location and data is now tracked and not lost.

FIG. 7 illustrates an example hospital tracking display 700 according to this disclosure. The hospital tracking display 700 can be displayed on the officer device 101 of FIG. 1 or another suitable computing device. The information collected and displayed in the hospital tracking display 700 is critical to life saving operations in a mass casualty event. Despite this fact, in most conventional systems, this information is collected manually using grease pencils and wipe boards.

The information in the hospital tracking display 700 can be automatically retrieved from different sources, such as populated patient tracking forms 400, and displayed automatically on the display 700. Using the hospital tracking display 700, medical ops officers, EMS teams, and other first responders are able to view and process data from the forms 400, which are submitted in real time. As data is submitted, the numbers and information in the hospital tracking display 700 are updated automatically with no extra writing or typing.

The “Red Beds” column 702 and “Yellow Beds” column 704 show the number of available beds of each type at each hospital or medical facility. Algorithms and counters capture patient status matched against the hospitals and then subtract that data from the available beds. When the number of available red or yellow beds at a particular facility decreases to zero, the corresponding cells in the columns 702, 704 can turn black to visually indicate no available beds.

The “Live Transit Time” column 706 displays the estimated transit time from the scene of the mass casualty event to the listed hospital based on route and live traffic calculations. Such live traffic data can be derived from one or more online data information repositories, such as GOOGLE ANALYTICS DATA, which is updated about every five minutes. In some embodiments, a geotag associated with the current location of a patient is provided to one or more mapping API's (e.g., GOOGLE MAPS API's) to determine the shortest travel time to a medical facility. In some embodiments, the times in the “Live Transit Time” column 706 are color-coded green to red based on the median travel time.

The hospital tracking display 700 also keeps a running number count of total patients, total patients in each color triage category, and total patients at each hospital. This is very useful data to the medical ops officer and critical to various command and control entities in the early phases of a mass casualty event.

The hospital tracking display 700 provides a number of technical advantages over conventional manual processes. For example, instead of pulling colored paper strips out of each hospitals “bucket” to count beds, the hospital tracking display 700 is populated automatically. Instead of accidently sending too many patients to a medical facility that is out of beds, the hospital tracking display 700 helps to prevent this by providing easy-to-see visual cues. Instead of requiring a user to manually wipe, erase, and update counts and numbers in the chaos and confusion of a mass casualty event (a process that is fraught with the possibility of error), the hospital tracking display 700 updates automatically, accurately, and in real time by retrieving data stored electronically (e.g., in the database 104). Instead of sending patients to any facility where an available bed exists (or even accidentally sending a patient to a facility where there are no available beds), the hospital tracking display 700 allows decisions to be made based on critical injuries and time to destination.

Indeed, many of the patient and hospital tracking processes described herein are performed as manual processes in conventional systems, using wipe boards and grease pencils or markers. However, such manual systems do not allow for collaborative viewing. That is, a wipe board at a mass casualty site cannot be viewed in real-time by EOC personnel at a different location. Also, other stakeholders, such as airline personnel, cannot view the wipe board in real-time. The disclosed embodiments allow all personnel in different groups to view information in real-time at different locations. Instead of EOC's and stakeholders constantly having to calling the medical ops officer for updates, these agencies can see the data remotely and in real time. Since all personnel can view information in real-time, further collaboration can occur between the informed groups in real-time. This is a technical advantage over conventional manual systems that simply come up short in the extremely time sensitive realm of a disaster.

Although FIGS. 2 through 7 illustrate various displays, forms, and user interfaces used in a process for patient tracking, various changes may be made to FIGS. 2 through 7. For example, the displayed information could include additional or alternative fields or be configured in any other suitable configuration(s). In some embodiments, various ones of the displays in FIGS. 2 through 7 can be formatted as separate tabs in a single application, which allows for quick and easy switching between different displays.

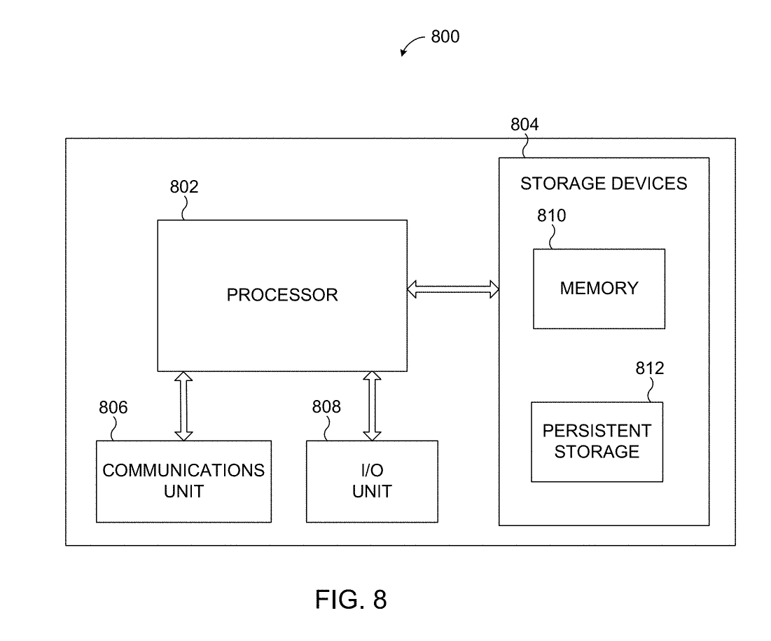

FIG. 8 illustrates an example device 800 for patient tracking during a mass casualty event according to this disclosure. The device 800 could, for example, represent any of the officer device(s) 101, a computing device at the medical facility 102, or the server 103. However, the device 800 could represent any other suitable device or components for patient tracking.

As shown in FIG. 8, the device 800 includes at least one processor 802, at least one storage device 804, at least one communications unit 806, and at least one input/output (I/O) unit 808. Each processor 802 can execute instructions, such as those that may be loaded into a memory 810. Each processor 802 denotes any suitable processing device, such as one or more microprocessors, microcontrollers, DSPs, FPGAs, ASICs, or discrete circuitry.

The memory 810 and a persistent storage 812 are examples of storage devices 804, which represent any structure(s) capable of storing and facilitating retrieval of information (such as data, program code, and/or other suitable information on a temporary or permanent basis). The memory 810 may represent a random access memory or any other suitable volatile or non-volatile storage device(s). The persistent storage 812 may contain one or more components or devices supporting longer-term storage of data, such as a read only memory, hard drive, Flash memory, or optical disc. In accordance with this disclosure, the memory 810 or the persistent storage 812 could be configured to store one or more algorithms that enable patient tracking.

The communications unit 806 supports communications with other systems or devices. For example, the communications unit 806 could include at least one network interface card or wireless transceiver facilitating communications over at least one wired or wireless network. As a particular example, in accordance with this disclosure, the communications unit 806 could include any suitable structure and components to support communication of patient tracking data with another component. The communications unit 806 may support communications through any suitable physical or wireless communication link(s).

The I/O unit 808 allows for input and output of data. For example, the I/O unit 808 may provide a connection for user input through a keyboard, mouse, keypad, touchscreen, or other suitable input device. The I/O unit 808 may also send output to a display, printer, or other suitable output device.

Although FIG. 8 illustrates one example of a device 800 for patient tracking, various changes may be made to FIG. 8. For example, various components in FIG. 8 could be combined, further subdivided, or omitted and additional components could be added according to particular needs. Also, computing devices can come in a wide variety of configurations, and FIG. 8 does not limit this disclosure to any particular configuration.

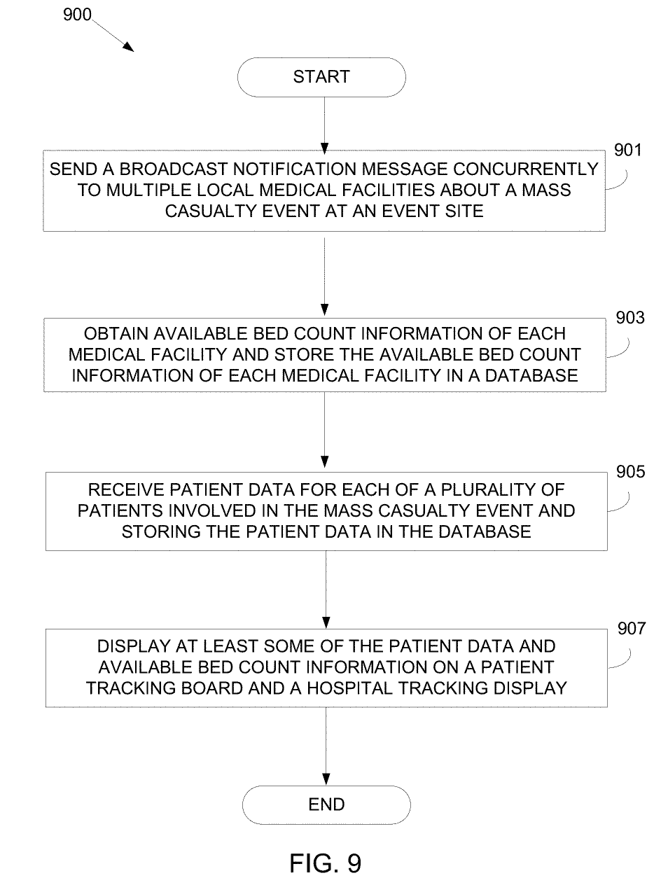

FIG. 9 illustrates an example method 900 for patient tracking during a mass casualty event according to this disclosure. For ease of explanation, the method 900 may be described as being performed using a computing device (such as the device 800 of FIG. 8 discussed below), and in accordance with the system 100 in FIG. 1 and the displays of FIGS. 2 through 7. However, the method 900 could be used with any suitable device, system, or display.

At step 901, a broadcast notification message is sent concurrently to multiple local medical facilities about a mass casualty event at an event site. Depending on the embodiment, the broadcast notification message can include a conference call or a textual network message.

At step 903, available bed count information is obtained from each of the multiple medical facilities in response to the broadcast notification message. The available bed count information of each medical facility is then stored in a database. In some embodiments, obtaining the available bed count information of the medical facility includes sending a hyperlink associated with a user interface to the medical facility, and receiving the available bed count information of the medical facility after a user associated with the medical facility actuates the hyperlink and inputs the available bed count information at the user interface. The available bed count information of each medical facility can be displayed at a user interface.

At step 905, patient data is received for each of a plurality of patients involved in the mass casualty event. The patient data is stored in the database. In some embodiments, receiving the patient data includes displaying a patient tracking form on a graphical display of each of multiple computing devices, and receiving user input of the patient data using the patient tracking form at each of the multiple computing devices.

At step 907, at least some of the patient data and at least some of the available bed count information is displayed on a patient tracking board and a hospital tracking display at a user interface.

Although FIG. 9 illustrates one example of a method 900 for patient tracking during a mass casualty event, various changes may be made to FIG. 9. For example, while shown as a series of steps, various steps shown in FIG. 9 could overlap, occur in parallel, occur in a different order, or occur multiple times. Moreover, some steps could be combined or removed and additional steps could be added according to particular needs.

It will be understood that the embodiments disclosed herein represent a system and method that can be one part of multi-platform solution. During a typical mass casualty event, some patients are able to walk away from the event site, some patients require treatment at triage or a hospital, and some patients may not survive. Different systems can support functions or operations for each of these groups of patients. Additional or alternative systems can support registration and information gathering for family members or loved ones of the patients. The disclosed system and processes can interface with these and other systems that provide for patient reception, family member reunification, and the like. All of these systems can be integrated to work together and share information.

In some embodiments, various functions described above are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

It may be advantageous to set forth definitions of certain words and phrases used throughout this patent document. The terms “application” and “program” refer to one or more computer programs, software components, sets of instructions, procedures, functions, objects, classes, instances, related data, or a portion thereof adapted for implementation in a suitable computer code (including source code, object code, or executable code). The terms “transmit” and “receive,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Definitions for other certain words and phrases are provided throughout this patent document. Those of ordinary skill in the art should understand that in many if not most instances, such definitions apply to prior as well as future uses of such defined words and phrases.

The description in the present application should not be read as implying that any particular element, step, or function is an essential or critical element that must be included in the claim scope. The scope of patented subject matter is defined only by the allowed claims. Moreover, none of the claims is intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” or “controller” within a claim is understood and intended to refer to structures known to those skilled in the relevant art, as further modified or enhanced by the features of the claims themselves, and is not intended to invoke 35 U.S.C. § 112(f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A method comprising:
sending a broadcast notification message concurrently to multiple local medical facilities about a mass casualty event at an event site;
obtaining, from each of the multiple medical facilities in response to the broadcast notification message, available bed count information of the medical facility and storing the available bed count information of each medical facility in a database;
receiving patient data for each of a plurality of patients involved in the mass casualty event and storing the patient data in the database; and
displaying at least some of the patient data and at least some of the available bed count information on a patient tracking board and a hospital tracking display at a user interface.

2. The method of claim 1, wherein obtaining the available bed count information of the medical facility comprises:
sending a hyperlink associated with a user interface to the medical facility; and
receiving the available bed count information of the medical facility after a user associated with the medical facility actuates the hyperlink and inputs the available bed count information at the user interface.

3. The method of claim 1, further comprising:
displaying the available bed count information of each medical facility at a user interface.

4. The method of claim 1, wherein the broadcast notification message comprises a conference call or a textual network message.

5. The method of claim 1, wherein receiving the patient data comprises:
displaying a patient tracking form on a graphical display of each of multiple computing devices; and
receiving user input of the patient data using the patient tracking form at each of the multiple computing devices.

6. The method of claim 5, wherein the patient tracking form is HIPAA compliant.

7. The method of claim 1, wherein the event site comprises a crash site, a government building, a shopping center, or a sports venue.

8. An apparatus comprising:
a display configured to show a user interface; and
at least one processing device configured to:
send a broadcast notification message concurrently to multiple local medical facilities about a mass casualty event at an event site;
obtain, from each of the multiple medical facilities in response to the broadcast notification message, available bed count information of the medical facility and store the available bed count information of each medical facility in a database;
receive patient data for each of a plurality of patients involved in the mass casualty event and store the patient data in the database; and
control the display to display at least some of the patient data and at least some of the available bed count information on a patient tracking board and a hospital tracking display at the user interface.

9. The apparatus of claim 8, wherein to obtain the available bed count information of the medical facility, the at least one processing device is configured to:
send a hyperlink associated with a user interface to the medical facility; and
receive the available bed count information of the medical facility after a user associated with the medical facility actuates the hyperlink and inputs the available bed count information at the user interface.

10. The apparatus of claim 8, wherein the at least one processing device is further configured to:
control the display to display the available bed count information of each medical facility at the user interface.

11. The apparatus of claim 8, wherein the broadcast notification message comprises a conference call or a textual network message.

12. The apparatus of claim 8, wherein to receive the patient data, the at least one processing device is configured to:
control the display to display a patient tracking form; and
receive user input of the patient data using the patient tracking form.

13. The apparatus of claim 12, wherein the patient tracking form is HIPAA compliant.

14. The apparatus of claim 8, wherein the event site comprises a crash site, a government building, a shopping center, or a sports venue.

15. A non-transitory computer readable medium containing instructions that when executed cause at least one processing device to:
send a broadcast notification message concurrently to multiple local medical facilities about a mass casualty event at an event site;
obtain, from each of the multiple medical facilities in response to the broadcast notification message, available bed count information of the medical facility and store the available bed count information of each medical facility in a database;
receive patient data for each of a plurality of patients involved in the mass casualty event and store the patient data in the database; and
control a display to display at least some of the patient data and at least some of the available bed count information on a patient tracking board and a hospital tracking display at a user interface.

16. The non-transitory computer readable medium of claim 15, wherein the instructions to obtain the available bed count information of the medical facility comprise instructions to:
send a hyperlink associated with a user interface to the medical facility; and
receive the available bed count information of the medical facility after a user associated with the medical facility actuates the hyperlink and inputs the available bed count information at the user interface.

17. The non-transitory computer readable medium of claim 15, further containing instructions that when executed cause the at least one processing device to:
control the display to display the available bed count information of each medical facility at the user interface.

18. The non-transitory computer readable medium of claim 15, wherein the broadcast notification message comprises a conference call or a textual network message.

19. The non-transitory computer readable medium of claim 15, wherein the instructions to receive the patient data comprise instructions to:
control the display to display a patient tracking form; and
receive user input of the patient data using the patient tracking form.

20. The non-transitory computer readable medium of claim 19, wherein the patient tracking form is HIPAA compliant.
